(19) United States
(12) Patent Application Publication
(10) Pub. No.: US 2006/0147493 A1
(43) Pub. Date: Jul. 6, 2006

(54) Packaging and dispensing of rapid dissolve dosage form

(76) Inventors: Robert K. Yang, Flushing, NY (US); Richard C Fuisz, McLean, VA (US); Garry L Myers, Kingsport, TN (US); Joseph M Fuisz, Washington, DC (US)

Correspondence Address: Daniel A Scola;Hoffmann & Baron, 6900 Jericho Turnpike, Syosset, NY, 11791, US

(21) Appl. No.: 10/521,823

(22) PCT Filed: Jul. 22, 2003
(86) PCT No.: PCT/US03/22882

§ 371 Date: Nov. 7, 2005

Related U.S. Application Data
(60) Provisional application No. 60/397,703, filed on Jul. 22, 2002.

(51) Int. Cl. A61K 47/00

(52) U.S. Cl. 424/439

(57) ABSTRACT

An oral dosage delivery vehicle comprising an edible film including a uniformly distributed active ingredient, wherein said film comprises dosage units releasably joined by one or more weakened sections, which permit said dosage units to be detached from said film.

### CROSS REFERENCE TO RELATED APPLICATIONS

This application is the National Stage of International Application No. PCT/US2003/022882, filed Jul. 22, 2003, which claims the benefit of U.S. Provisional Application No. 60/397,703, filed Jul. 22, 2002, the contents of which are incorporated by reference herein.

### FIELD OF THE INVENTION

The present invention relates to devices and methods for the storage and dispensing of an edible, thin, water-soluble, rapid dissolve dosage form.

### BACKGROUND OF RELATED TECHNOLOGY

Water-soluble thin films have recently become very popular as a form of breath freshener. These films generally include a breath freshening agent in a polymer film. These films have a convenient small size which contributes to their popularity.

However, such films and their current packaging have several disadvantages. The films themselves are typically too thin to support other active ingredients, such as a pharmaceutical active. In addition, the packaging does not provide an effective air/moisture barrier. The result is a film that frequently dries out, becoming too brittle for use. Furthermore, when the films are placed in the packaging, they are usually stacked, and frequently adhere to an adjacent film. Therefore, a person attempting to remove a single film from the packaging may inadvertently remove two or more.

It is desirable to provide a film and packaging that includes a barrier to moisture, air, and light, which can interfere with the quality of the film product and the active ingredients contained within the film. Ideally, this film will be capable of supporting not only a breath freshener as the active, but also pharmaceutical products. It is further desirable to provide a method of dispensing the films, wherein only the desired number of films may be removed at a time.

### SUMMARY OF THE INVENTION

The present invention provides an oral dosage delivery vehicle including an edible film having a sheet-like construction, wherein the film comprises dosage units releasably joined by one or more weakened sections, which permit said dosage units to be detached from the film. Desirably the weakened sections include a perforated or scored configuration that may be cut into the film. More desirably, the individual dosage units are uniform in their composition and include a uniformly distributed active ingredient, such as a drug, cosmetic, or bioactive agent and the like.

The present invention also provides a package for the storage and dispensing of an edible sheet-like rapid dissolve dosage form, such as a thin film. The invention includes a pouch into which a sheet-like dosage form has been placed. The pouch includes top and bottom layers that are sealed at the edges leaving a space between them for storing the sheet-like dosage form. The pouch may also include a means for resealing to prevent additional introduction of light, moisture and/or air. Such resealing means may include adhesive coatings, mechanical closures, such as those found on resealable plastic bags and other similar resealing means.

While a variety of film-forming techniques may be used to produce the rapid dissolving sheet-like dosage forms of the present invention, the present invention also includes a unique method of producing the edible dosage forms such that uniform distribution of the compositional components are evenly distributed throughout the film. This process is described in detail in co-pending U.S. patent application Ser. No. 10/074,272, entitled “Thin Film with Non-Self-Aggregating Uniform Heterogeneity and Drug Delivery Systems Made Therefrom”, the subject matter of which is herein incorporated by its entirety. The process provides dosage forms that consistently include substantially the same amount of the active ingredient and may include a wide variety of active ingredients, including pharmaceutical actives.

A further aspect of the present invention provides a method of storing an edible sheet-like rapid dissolve dosage form. The method includes first preparing a sheet-like dosage form that includes one or more unit doses of an active ingredient. The sheet may be separated by weakened sections between individual units or segments, desirably of substantially equal area, which represent individual dosage units or a predetermined fraction of a dosage unit. The sheet is placed into a pouch that has top and bottom layers, which desirably include a barrier layer. The top and bottom layers are sealed at the edges, which may be through the use of an adhesive, desirably, one that is heat sealable, pressure-sensitive or which forms a bond at room temperature.

A still further aspect of the present invention provides a method of dispensing a sheet-like dosage form. First, a sheet-like dosage form that includes one or more doses of an active ingredient is prepared. Then the dosage form may be separated at weakened sections therein to form individual segments or units, desirably of substantially equal area, representing either an individual dosage unit or a fraction thereof, as described above. The sheet is placed in a pouch having top and bottom layers that may include a barrier material. The pouch is sealed at the edges. The dosage form is dispensed by opening a portion of the pouch, desirably a portion of the edge, then separating one or more sections of the dosage form from the sheet along the weakened sections, and removing the separated dosage unit(s) from the pouch. The pouch may then be resealed.

### BRIEF DESCRIPTION OF THE DRAWINGS

FIG. 1-FIG. 3 are perspective views of the sheet-like dosage forms of the present invention.

FIG. 4 is a perspective view of the unassembled top and bottom sheets including the sheet-like dosage form.

FIG. 5 is a perspective view of the assembled packaging of the present invention.

FIG. 6 is a cross-section of a laminate that may be used as the top or bottom layer of the packaging.

FIG. 7-FIG. 9 and FIG. 9a are cross-sections of the packaging along line 20-20, including a resealable edge.

FIG. 10 is a cross-section of the packaging along line 20-20, showing the inclusion of more than one active-containing sheet.

FIG. 11 is a topside view of the dosage form showing details of a weakened section.

FIG. 12-FIG. 13 are cross-sections of the dosage form along line 50-50, showing, in detail, different configurations of the weakened section.

FIG. 14 is a topside view of the dosage form showing details of a weakened section.

FIG. 15 is a side view of the detail of a weakened section.

FIG. 16-FIG. 17 are side views of the dosage form including a backing layer.

FIG. 18 is a perspective view of a film that includes a surface altered to increase the surface area.

FIG. 19-FIG. 21 are cross-sections along line 60-60 showing the detail of the surface alteration.

### DETAILED DESCRIPTION OF THE INVENTION

The present invention includes a “sheet-like” dosage form or a film. For the purposes of this invention the term “sheet-like” dosage form, “sheet,” and film are meant to include a water soluble delivery system having a thickness of less than about 15 mils. The sheet-like dosage forms are desirably edible and contain an active ingredient.

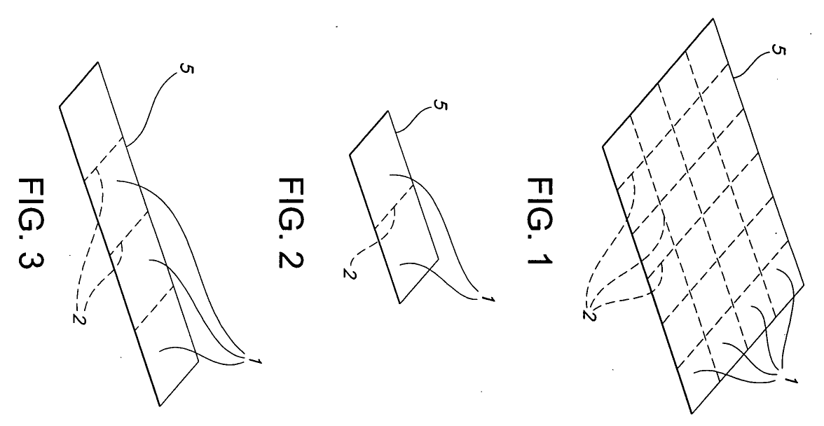

The sheets or films 5 of the water-soluble rapid dissolve dosage forms, which may include one or more dosage forms 1, may include weakened sections, as shown in FIG. 1-3. The weakened section is designed such that application of a bending force thereat breaks apart adjacent units from each other. The weakened sections 2 in the sheet may form straight lines in either a vertical direction or a horizontal direction, or combinations of both, although lines which are not straight may also be used. The weakened sections will divide the sheet into segments that represent individual doses of an active. The segments may be in a variety of different shapes and sizes including square, rectangle, triangle, trapezoid, circle, ellipse, etc. Desirably, the lines will combine to provide sections of substantially equal area. Each section will represent either an individual dosage form or a predetermined fraction of a dosage form. For example, a child dose may be one-half of an adult dose for a particular active, or where a large film is required for a particular dose, the film may be divided to provide ease of administration. For example, in FIG. 2, which shows a sheet perforated into two sections, each section may be a dosage unit, or the entire sheet may be a single dosage unit.

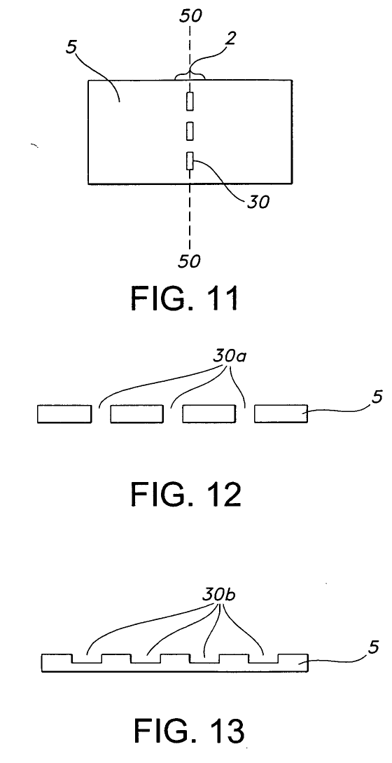

The weakened sections themselves may take on a variety of different configurations, as shown in FIGS. 11-13. In general, a weakened section of the sheet is a location of the sheet that has been altered to permit separation of sections of the film. Desirably, the weakened sections are formed so that the individual dosage units are of substantially identical dimensions. Where the dosage form includes an active, there is uniformity among dosage units that have been separated, desirably where there is less than 10% variance among the individual dosage units both before and after separation. The weakened sections may be in any configuration that permits one section of the film to be separated from the remaining film. Examples of weakened sections include perforations or scored areas that form voids 30 in the material, as in FIG. 11. As shown in FIG. 12 and FIG. 13, the voids 30a and 30b, respectively, may either completely or partially penetrate the film.

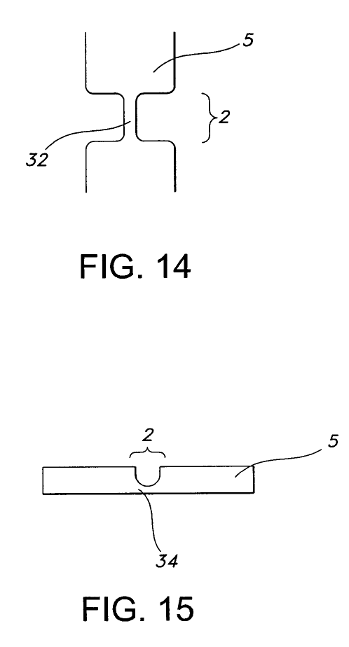

Other examples of weakened sections may be used when unifonrmity of film size is not an issue, including where the dosage form does not incorporate a drug active. Examples include narrower sections 32 of the film 5, as shown in FIG. 14, and areas that have less thickness 34 than the surrounding film, as shown in FIG. 15. The weakened sections may be formed by cutting the film, by casting the film into a pre-determined shape, or by casting the film onto a patterned surface that results specifically selected thinner areas of the film.

The weakened sections, described above, serve as break-points where the film is intended to be separated into individual dosage units. The film itself is generally flexible to avoid inadvertent or premature separation and breaking of dosage units. However, the weakened sections allow the film to be separated at pre-determined segments that will represent the individual doses. This separation may be by breaking, bending, tearing, or otherwise detaching the individual segments the film or sheet-like construction. The dosage forms may include a line formed of an edible ink along the weakened sections. Such a line serves as a “safety” indication line to visibly indicate to the user that they have broken off the appropriate dosage unit. Particularly where a perforated dosage form is used, this will assure that the user has detached the dosage form at the appropriate location. This may be accomplished by perforating the film with a serrated knife-like instrument, which may include edible ink liners that mark the film as it is cut. Other methods of forming the safety line, such as printing, may be used.

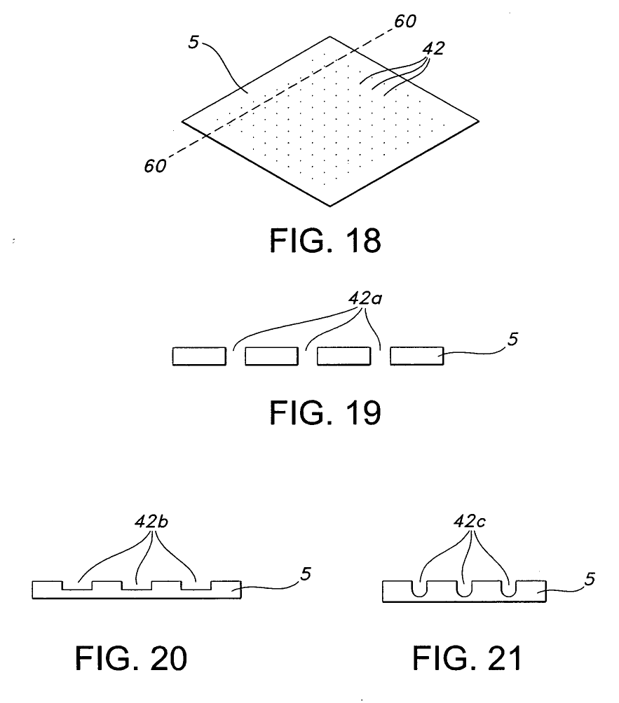

The surface of the film may either be smooth, or altered in a way to increase the surface area of the film. Where the surface area is altered, it may include voids 42 or holes as shown in FIG. 18. The voids will be placed in the film in a uniform manner, which does not affect the uniformity of the distribution of any active that the film may contain. The effect of the voids is an increase in the surface area of the film, which will speed the dissolution time of the film when administered, including an increased speed of dissolution of the film in the mouth. As shown in FIGS. 19-21, the voids may take a variety of different shapes, and may go either completely through the depth of the material as the voids 42a of FIG. 19 or partially through as the voids 42b and 42c, of FIGS. 20-21, respectively. The alteration of the surface area of the film will be conducted in such a way that will maintain the strength of the film, unlike the alteration at the weakened sections. Ideally, the voids are formed by either cutting the film or by casting the film on a template to produce a specific pattern.

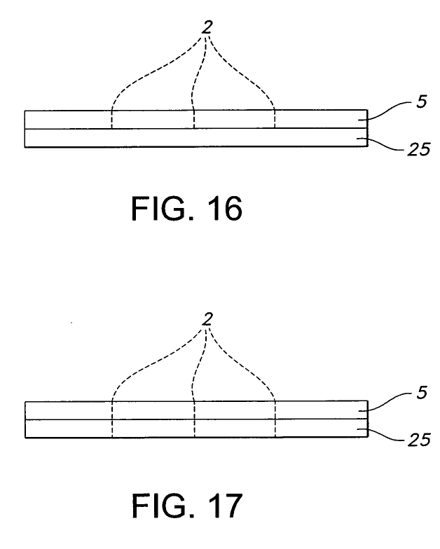

As shown in FIGS. 16 and 17, the sheet-like dosage form 5 may include a substantially water-insoluble backing layer 25. While the films 5 may be self-supporting, the second carrier layer 25 may act as a support layer for the dosage form 5. The carrier layer 25 may either be continuous, without the inclusion of weakened sections 2 as in FIG. 16, or it may include weakened sections 2a corresponding to the weakened sections 2 of the film 5 to allow the second layer 25 to separated along with a corresponding section of the dosage form 5, as shown in FIG. 17.

The edible sheet-like dosage forms of the present invention include a water-soluble polymer. Useful water-soluble polymers for the present invention include cellulosic materials, gums, proteins, starches, and combinations thereof.

One advantage of the present invention is that dosage units or a portion thereof may easily be dispensed. For example, a 10 mg dosage unit may itself contain a break-point section, i.e., a perforated section, to allow the patient to divide the taking of the required 10 mg dose over two different time intervals, or to simply make it easier to ingest at one time.

Examples of cellulosic materials include, without limitation, carboxymethyl cellulose, hydroxyl methyl cellulose, hydroxyethyl cellulose, hydroxypropyl cellulose hydroxypropylmethyl cellulose, and combinations thereof.

Examples of water-soluble gums include gum arabic, xanthan gum, tragacanth, acacia, carageenan, guar gum, locust bean gum, pectin, alginates and combinations thereof.

Examples of other polymeric materials include polyvinyl alcohol, polyacrylic acid, polyvinyl pyrrolidone, poly(meth)acrylate, poly(meth)copolymers and combinations thereof.

Useful starches include gelatinized, modified or unmodified starches. The source of the starches may vary and include tapioca, rice, corn, potato, wheat and combinations thereof.

Useful water-soluble protein polymers gelatin, zein, gluten, soy protein, soy protein isolate, whey protein, whey protein isolate, casein, levin, collagen and combinations thereof.

Additional water-soluble polymers include dextrin, dextran and combinations thereof, as well as chitin, chitosin or combinations thereof, and polydextrose.

The sheet-like dosage forms of the present invention further include an active component selected from cosmetic agents, pharmaceutical agents, bioactive agents, including antigens, such as ragweed pollen, and combinations thereof. The active component may be present in any amount effective for the intended treatment. It is particularly desirable and an advantage of the present invention that the active component can be included in high loads. For example, the active component may be present in amounts up to about 60% by weight of the total composition and desirably in amounts of 0.01% to about 50% by weight of total composition.

The active components that may be incorporated into the films of the present invention include, without limitation, medicaments, flavors, fragrances, enzymes, preservatives, sweetening agents, colorants, spices, vitamins and combinations thereof.

A wide variety of medicaments and pharmaceutical compositions may be included in the dosage forms of the present invention. Examples of useful drugs include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, aaino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

The dosage forms of the present invention further includes one or more members selected from taste-masking agents, plasticizing agents, surfactants, emulsifying agents, thickening agents, binding agents, cooling agents, saliva-stimulating agents, sweetening agents, antimicrobial agents, antigens and combinations thereof.

### EXAMPLES

Water soluble thin film compositions useful in the present invention are prepared using the amounts described in Table 1.

TABLE 1

| | WEIGHT % | | | | | | | | | |
|---|---|---|---|---|---|---|---|---|---|---|
| Ingredient | A | B | C | D | E | F | G | H | I | J |
| Hydroxypropylmethyl | 4.03 | 3.77 | 3.70 | 3.84 | 0 | 3.67 | 4.03 | 0 | 6.24 | 6.24 |
| cellulose | | | | | | | | | | |
| Peppermint oil | 2.94 | 1.93 | 2.39 | 0 | 0 | 2.67 | 2.94 | 2.67 | 4.17 | 0 |
| Sweetener | 2.20 | 0.32 | 0.23 | 0 | 0.17 | 1.53 | 2.20 | 1.54 | 3.34 | 3.34 |
| Polyvinylpyrrolidone | 2.68 | 2.01 | 2.39 | 0 | 0 | 2.33 | 2.68 | 2.34 | 4.16 | 4.16 |
| Tween 80¹ | 2.24 | 1.07 | 1.48 | 1.42 | 0.55 | 1.35 | 2.24 | 0 | 0 | 0 |
| Simethicone² | 0.66 | 0.42 | 0.68 | 0.22 | 0.22 | 5.00 | 2.00 | 0 | 0.98 | 0.98 |
| Listerine³ | 0 | 0 | 0 | 0 | 92.41 | 0 | 0 | 0 | 0 | 0 |
| Raspberry flavor | 0 | 0 | 0 | 0 | 0 | 0 | 0 | 0 | 0 | 0.12 |
| Cornstarch⁴ | 2.68 | 0 | 0 | 0 | 0 | 0 | 2.68 | 0 | 4.16 | 4.16 |
| Water | 73.53 | 90.47 | 89.14 | 92.22 | 0 | 83.45 | 72.19 | 93.46 | 62.15 | 60.00 |
| Loratadine⁵ | 4.29 | 0 | 0 | 2.31 | 0 | 0 | 4.29 | 0 | 6.65 | 0 |
| Pullulan⁶ | 0 | 0 | 0 | 0 | 6.65 | 0 | 0 | 0 | 0 | 0 |
| Calcium Carbonate | 1.43 | 0 | 0 | 0 | 0 | 0 | 1.43 | 0 | 2.22 | 12.15 |
| Xanthan Gum | 0.30 | 0 | 0 | 0 | 0 | 0 | 0.30 | 0 | 0.46 | 0 |
| Propylene Glycol | 3.02 | 0 | 0 | 0 | 0 | 0 | 3.02 | 0 | 4.67 | 8.84 |
| Ethoxylated castor oil⁷ | 0 | 0 | 0 | 0 | 0 | 0 | 0 | 0 | 0.80 | 0 |

¹Available from ICI Americas
²Available from OSI
³Available from Pfizer, Inc. including thymol (0.064%), eucalyptol (0.092%), methyl salicylate (0.060%), menthol (0.042%), water (up to 72.8%), alcohol (26.9%), benzoic acid, poloxamer 407, sodium benzoate, and caramel color
⁴Available from Grain Processing Corporation as Pure Cote B792
⁵Available from Schering Corporation as Claritin
⁶Available from Hayashibara Biochemical Laboratories, Inc., Japan
⁷Available as Cremophor EL from BASF

The ingredients of inventive compositions A-J were combined by mixing until a uniform mixture was achieved. Vacuum was then applied over 20 min. starting at 500 mmHg and ending at 660 mmHg until all air was removed from the suspension. The compositions were then cast onto a silicone coated paper using a 200 micron spiral wound rod and a K control Coater Model 101 (RK Print Coat Inst. Ltd.). These films were then dried by applying heat at 90° C. to the bottom of the film. No external thermal air currents were present above the film. The films were dried to less than about 6% by weight water in about 4 to 6 minutes. The films were flexible, self-supporting and provided a uniform distribution of the components within the film.

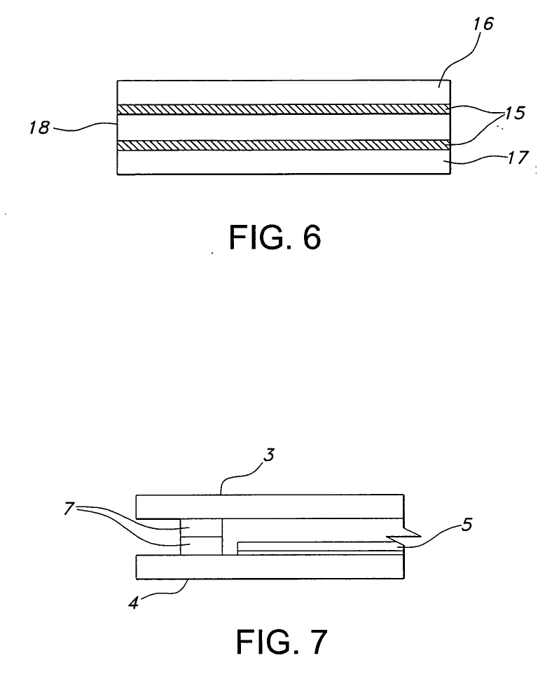

The layers that form the pouch of the present invention may be made of a variety of different materials and constructions. As shown in FIG. 6, may themselves include one or more layers that are laminated together with an adhesive. A variety of different materials may be used for each of the layers. Desirably, the top and bottom layers will each include a laminate of at least two layers. More desirably, a three-layer laminate will be included. The three layer laminate will include an outer layer 16, an inner layer 17, and an intermediate layer 18 that include an adhesive 15 dispersed therebetween.

One effective barrier material is a metal foil, such as aluminum, which provides a barrier to light, moisture and air. Depending on the barrier requirements of the film and any active contained therein, other materials may be selected for the various layers. These materials may include paper, polyolefins, such as polyethylene or polypropylene, polyester, hydrolyzed polyvinyl acetate co-polymer and blends thereof.

The layers may also include, where necessary, an anti-static agent, anti-fogging agent, ultraviolet light absorber, antioxidant, plasticizer, lubricant, nucleating agent, dispersant, colorant, anti-fungus agent, anti-microbial agents, inorganic filler, and the like.

In one aspect of the invention, the layers may be laminated together by first dispersing an adhesive between the layers which may then be co-extruded to form the multi-layered packaging of the present invention. The adhesive may include polyolefin resins such as those modified with unsaturated carboxylic acid or a derivative thereof. The unsaturated carboxylic acid may include, without limitation acrylic acid, methacrylic acid, maleic acid, funaric acid, crotonic acid, itaconic acid, citraconic acid and the like, as well as esters and anhydrides thereof.

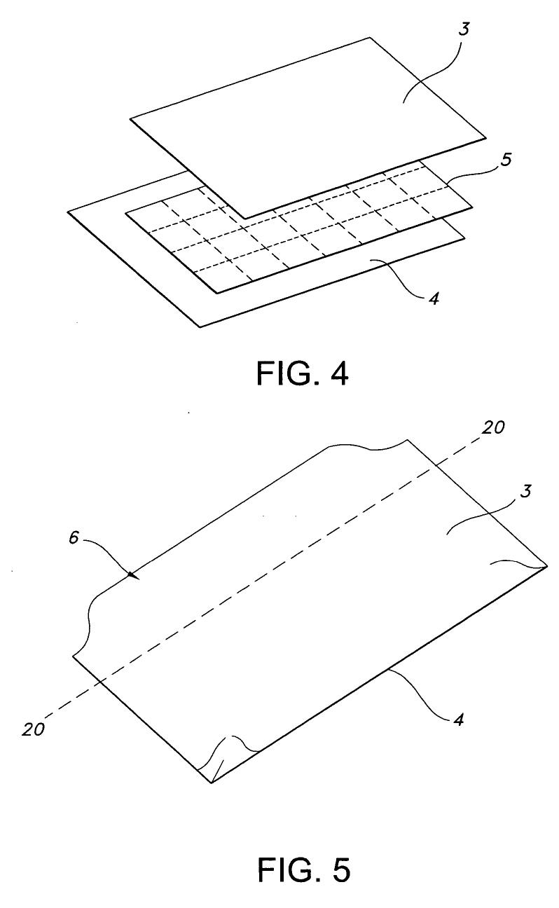

FIG. 4 shows layers of a pouch containing the sheet-like dosage forms 5. The pouch includes top 3 and bottom 4 layers, which may each include a multi-layered laminate material, which is sealed along the perimeter as shown in FIG. 5. This may be accomplished, for example, by heat sealing or with an adhesive, such as a pressure-sensitive adhesive.

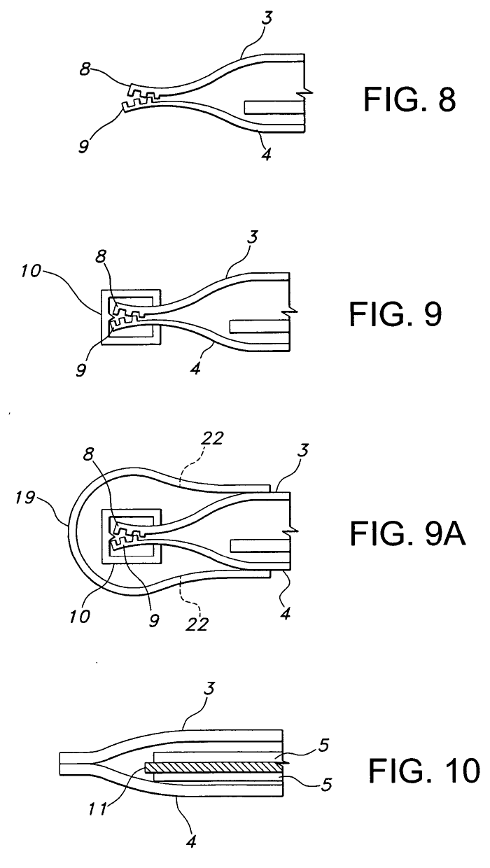

The resulting pouch may also be resealable along a portion of its perimeter as shown in FIGS. 7-9 and 9a. As shown in FIG. 7, this is accomplished by providing a portion of the perimeter with a pressure sensitive adhesive 7 between the top 3 and bottom 4 layers. At this point along the perimeter, the top and bottom layers may be separated to allow removal of a dosage form and then resealing. Alternatively, as shown in FIGS. 8 and 9, a portion of the perimeter may be sealed and resealed by forming a zipper track 8 along the top layer 3 and a corresponding track 9 along the bottom layer 4 of a portion of the pouch. The two tracks should be formed such that they can engage, forming a seal along a portion of the perimeter. The track may be engaged by either manual pressure, or by the use of a zipper 10. FIG. 9a adds a “tamper resistant” feature to the packaging, where another layer of material 19 is included which covers the resealable zipper. This may either be attached by the use of an adhesive to the top 3 and bottom 4 layers, or alternatively may form an additional layer over the length of the top 3 and bottom 4 layers (not shown). This allows the pouch to be opened by the consumer by first removing the surrounding layer 19, desirably at a perforated section 22 to permit access to the resealable opening.

More than one sheet of the dosage form may be included within the pouch. For example, two or more sheets may be stacked on top of each other. As shown in FIG. 10, a non-water soluble support film 11, such as a layer of polyolefin, also in the form of a sheet, may be placed between the sheets of water-soluble dosage forms to prevent the dosage 5 forms in the two or more sheets from adhering to each other.

To dispense a dosage form, the pouch is first opened. This is by either by tearing the pouch open or by separating the top and bottom layers of the pouch. The sheet may either be removed from the pouch, or desirably, where the top and bottom layers are separated, the film is presented apart from the top and bottom pouch layers, which are peeled away, to provide greater ease in dispensing. In one embodiment, the top and bottom pouch layers are peeled apart at one end to present the dosage form in an erect or vertical position for easy handling. Then, the unit sections of the film/dosage form may be separated by tearing along the weakened or perforated sections to separate a dose, or fraction thereof, from the sheet. The remainder of the sheet may then be returned to the pouch until a future dose is needed.

While there have been described what are presently believed to be the certain desirable embodiments of the invention, those skilled in the art will realize that changes and modifications may be made thereto without departing from the spirit of the invention, and it is intended to include all such changes and modifications as fall within the true scope of the invention.

What is claimed is:

1. An oral dosage delivery vehicle comprising an edible film including a uniformly distributed active ingredient, wherein said film comprises dosage units releasably joined by one or more weakened sections, which permit said dosage units to be detached from said film.

2. The delivery vehicle of claim 1, wherein said film is self-supporting.

3. The delivery vehicle of claim 1, wherein said weakened sections are perforations.

4. The delivery vehicle of claim 1, wherein said weakened sections are scored.

5. The delivery vehicle of claim 1, wherein said active is a pharmaceutical or cosmetic active.

6. The delivery vehicle of claim 1, wherein said active varies no more than 10% among said dosage units.

7. The delivery vehicle of claim 1, wherein said film includes a carrier layer of a substantially water insoluble material.

8. The delivery vehicle of claim 1, wherein the film has a surface with a surface area comprising voids that increase the surface area.

9. The delivery vehicle of claim 8, wherein said voids increase the speed of dissolution of said delivery vehicle.

10. The delivery vehicle of claim 8, wherein the voids are selected from the group consisting of voids that go through the entire depth of the film, voids that go through a portion of the depth of the film, and voids that are formed by casting the film on a patterned template.

11. An oral dosage delivery vehicle comprising an edible film, wherein said film comprises dosage units releasably joined by one or more weakened sections, which permit said dosage units to be detached from said film.

12. The delivery vehicle of claim 11, wherein said weakened sections contain less film composition than surrounding areas.

13. The delivery vehicle of claim 11, wherein said film is self-supporting.

14. The delivery vehicle of claim 11, wherein said weakened sections are perforations.

15. The delivery vehicle of claim 11, wherein said weakened sections are scored.

16. The delivery vehicle of claim 11, further comprising an active ingredient.

17. The delivery vehicle of claim 16, wherein said active is uniformly distributed.

18. The delivery vehicle of claim 11, wherein said film includes a carrier layer of a substantially water insoluble material.

19. The delivery vehicle of claim 17, wherein the film has a surface with a surface area comprising voids that increase the surface area.

20. The delivery vehicle of claim 19, wherein the voids are selected from the group consisting of voids that go through the entire depth of the film, voids that go through a portion of the depth of the film, and voids that are formed by casting the film on a patterned template.

21. A package for the storage and dispensing of a sheet-like rapid dissolve dosage form, comprising:
a) a pouch comprising a top layer and a bottom layer each having an outer edge, wherein said top and bottom layers are sealed at the respective outer edges to define an enclosed interior surface therewithin, and
b) a sheet-like rapid dissolve dosage form contained within said enclosed space.

22. The package of claim 21, wherein said dosage form includes an active ingredient and comprises a sheet of one or more dosage forms.

23. The package of claim 22, wherein said dosage form includes weakened sections.

24. The package of claim 22, wherein said weakened sections include perforations.

25. The package of claim 23, wherein said weakened sections form one or more equally spaced lines in a direction selected from vertical, horizontal, and combinations thereof.

26. The package of claim 21, wherein said pouch is resealable.

27. A method of storing a sheet-like rapid dissolve dosage form comprising the steps of:
a) preparing a sheet-like dosage form that includes an active ingredient;
b) defining said dosage form into a plurality of substantially equal weakened sections separable dosage units by forming in said dosage form;
c) preparing a pouch comprising top and bottom layers each having an outer edge;
d) placing said dosage form between said top and bottom layers; and
e) sealing the outer edges of said pouch.

28. A method of dispensing a sheet-like dosage form comprising the steps of:
a) preparing a sheet-like dosage form that includes an active ingredient;
b) separating said dosage form by weakened sections into segments of substantially equal area;
c) preparing a pouch comprising top and bottom layers each of said layers having an edge surrounding the circumference of said layer;
d) placing said dosage form between said top and bottom layers;
e) separably sealing a portion of said edges of said pouch;
f) opening a portion of said pouch;
g) opening one or more of said sections of said dosage form along said perforations; and
h) removing said one or more segments from said pouch.
